(19) United States
(12) Patent Application Publication
(10) Pub. No.: US 2005/0015072 A1
(43) Pub. Date: Jan. 20, 2005

(54) Cannula having buckle resistant apertures

(75) Inventors: Rebecca L. Engel, Kalamazoo, MI (US); Donald R. Sandmore, Newaygo, MI (US); David Weston, Rockford, MI (US); Frederick A. Shorey, East Grand Rapids, MI (US); David B. DeWindt, Grand Rapids, MI (US)

Correspondence Address: FOLEY & LARDNER, 777 EAST WISCONSIN AVENUE, SUITE 3800, MILWAUKEE, WI, 53202-5308, US

(73) Assignee: Medtronic, Inc.

(21) Appl. No.: 10/619,932

(22) Filed: Jul. 15, 2003

(51) Int. Cl. A61M 25/00

(52) U.S. Cl. 604/523

(57) ABSTRACT

A cannula has a body with a proximal end and a distal end. The body has a wall defining a lumen extending from the proximal end to the distal end, the lumen having a longitudinal axis. The cannula further has a plurality of apertures in the wall that are interconnected with the lumen, each of the apertures having a longer major axis and a shorter minor axis. The longer major axis of the apertures is perpendicular to the longitudinal axis of the lumen.

### FIELD OF THE INVENTION

The present invention relates to a medical cannula. In particular, the present invention relates to a cannula having apertures that are buckle resistant.

### BACKGROUND OF THE INVENTION

During cardiac surgery, circulation of blood through a patient's body may be maintained by connecting the patient to an extracorporeal system, such as a heart-lung machine. The heart-lung machine adds oxygen to and removes carbon dioxide from the blood, heats or cools the blood, and provides impetus to the blood to cause the blood to circulate through the patient's vascular system.

Connecting a patient to an extracorporeal system is typically done by inserting a cannula into the patient's venous system near or in the heart to remove blood from the patient and direct it to the extracorporeal circuit. After the blood has passed through the extracorporeal circuit, the blood is infused into the patient's arterial system near the heart.

The venous cannula that is inserted into the heart to siphon blood away for entry into the heart-lung machine is typically inserted into the right atrium and/or vena cava. The venous cannula may be a single stage device having one set of input apertures or a two-stage device used to simultaneously drain the right atrium and superior vena cava through an atrial basket while the inferior vena cava is drained through another set of apertures at the distal tip of the cannula. Oxygenated blood is returned to the heart from the heart-lung machine using an arterial cannula to return blood to the aorta.

Regardless of the type of surgical procedure in which a cannula is being used, a cannula may have to be flexed or bent as it is inserted into the proper location in a patient's body. Whether the cannula is being used to drain fluids or to insert fluids, it is desirable to maintain proper fluid flow through the cannula at all times. Accordingly, cannula designs attempt to minimize kinking of the tube. Kinking of a cannula occurs when a tube is flexed and results in the sides of the tube touching each other and folding in half, thus blocking or minimizing fluid flow through the interior lumen. Cannula materials, design, and aperture placement are chosen to minimizing such kinking. A common approach is to utilize a reinforcing spring integrated into the walls of the cannula to prevent collapse of the lumen when the cannula is flexed.

Another challenge of cannula design is the minimization of buckling of the apertures in the walls of the cannula. Typically apertures are punched or drilled into the walls of a cannula to permit flow into or out of the lumen. Many apertures may be used in order to improve the drainage or perfusion characteristics of the cannula. When the cannula is flexed during placement of the cannula into the body, such as when inserting a cannula into the inferior vena cava or right atrium, the apertures may buckle. Buckling is the phenomenon of the sides of individual cannula apertures puckering outward when the cannula body is flexed.

Referring to FIGS. 1 and 2, when a cannula 1 is flexed, aperture buckling can occur. The sides 3, 4 of individual apertures 5 on the concave side 8 are necessarily pushed toward one another as the cannula 1 is bent. As the sides 3, 4 close toward one another, the apertures 5 may buckle outward at other sides 6, 7.

The buckling phenomenon is undesirable because the portion of the aperture that buckles outward creates a scoop that extends outward from the cannula wall and may damage the sides of a vessel wall in the patient. For example, an arterial cannula must be flexed as it is guided around the aorta when performing a cardiopulmonary bypass procedure. It is desirable to minimize tissue damage to the internal aorta walls due to the puckering of apertures in the cannula as the cannula is placed into position.

Conventional cannula designs attempt to minimize kinking of the cannula and buckling of flow apertures through the use of different materials, such as the use of a hard plastic insert in the cannula that contains the flow apertures and a helical reinforcing spring to increase kink resistance. However, it is desirable to enhance cannula flexibility while also minimizing kinking of the cannula and buckling of the cannula apertures. While a reinforcing wire may aid in preventing kinking of the cannula, it may be desirable to omit the reinforcing wire at the distal end of the cannula where the flow apertures reside, thus requiring another solution to the design challenges at the distal end of the cannula. Further, it is desirable to maintain similar flow characteristics through the flow apertures while minimizing the chances of the apertures buckling when the cannula is bent or flexed.

There is a need for a cannula design that is flexible yet resistant to kinking. Further, there is a need for a cannula having flow apertures that resist buckling when the cannula is flexed. It would be desirable for a cannula design or method of cannula manufacture to provide one or more of these or other advantageous features. Other features and advantages will be made apparent from the present specification. The teachings disclosed extend to those embodiments that fall within the scope of the appended claims, regardless of whether they accomplish one or more of the aforementioned needs.

### SUMMARY OF THE INVENTION

The invention relates to a cannula having a body with a proximal end and a distal end. The body has a wall defining a lumen extending from the proximal end to the distal end. The lumen has a longitudinal axis and a plurality of apertures in the wall interconnected with the lumen. Each of the apertures has a longer major axis and a shorter minor axis and the longer major axis is perpendicular to the longitudinal axis of the lumen.

The invention further relates to a cannula having a body with a proximal end and a distal end, the body having a wall defining a lumen extending from the proximal end to the distal end. The cannula further has a plurality of eye-shaped apertures in the wall.

Further still, the invention relates to a method of making a cannula. The method includes the steps of forming a cannula body having a wall defining a lumen and bending the cannula body in a first direction such that the cannula body has a concave side and a convex side. The method further includes the steps of punching an aperture into the concave side of the body using an oval punch and then straightening the cannula body.

The invention is capable of other embodiments and of being practiced or being carried out in various ways. Alternative exemplary embodiments relate to other features and combinations of features as may be generally recited in the claims.

### BRIEF DESCRIPTION OF THE DRAWINGS

The invention will become more fully understood from the following description, taken in conjunction with the accompanying drawings, wherein like reference numerals refer to like elements, in which:

FIG. 1 is a perspective view of a conventional cannula in a flexed configuration;

FIG. 2 is a sectional view taken generally along line 2-2 of FIG. 1;

FIG. 3 is an elevation view of a cannula according to a first embodiment of the invention;

FIG. 4 is an enlarged detail elevation view of a segment of the distal end of the cannula shown in FIG. 2, the segment location generally indicated by line 4-4 of FIG. 3;

FIG. 5 is a perspective view of the distal end of the cannula of FIG. 3, shown in a flexed configuration;

FIG. 6 is an elevation view of the cannula of FIG. 2, shown in a flexed configuration;

FIG. 7 is a sectional view taken generally along line 7-7 of FIG. 5; and

FIG. 8 is an elevation view of a cannula according to a second embodiment of the invention.

### DETAILED DESCRIPTION OF PREFERRED EMBODIMENTS

Referring to FIG. 3, a catheter or cannula, shown as, but not limited to, venous cannula 10 has a body with a proximal end 12 and a distal end 14. A tip 16 is located at the distal end 14 of the cannula 10 and a lumen 18 defined by a wall 20 extends through the cannula 10 from the proximal end 12 to the tip 16.

The lumen 18 may be open at the proximal end 12 to be connected to a cardiac bypass system such as a heart-lung machine. The distal end 14 includes a number of holes or apertures 22 for draining blood from the heart to pass through the lumen 18 and into a heart-lung machine. Further, a distal aperture 24 may be provided at the tip 16 of cannula 10. Various methods of performing a cardiopulmonary bypass are known in the art. In the embodiment depicted in FIG. 3, the cannula is a single stage venous cannula. In other embodiments, the cannula may be of other types, such as a dual stage venous cannula having two sets of apertures used to drain two portions of the heart simultaneously.

Further referring to FIG. 3, a reinforcement member, shown as helical reinforcement spring 26 may extend over a substantial portion of a length of cannula 10 to prevent kinking or closing off of the lumen 18 when the device is flexed, bent, or otherwise manipulated when in use by a surgeon.

Referring to FIG. 4, one or more apertures 22 may be eye-shaped. As defined herein, eye-shaped means aperture 22 is defined by first arcuate portion 30 and second arcuate portion 32 that intersect with one another at two tips or corners 34, 36. Aperture 22 has a longer major axis 38 and a shorter minor axis 40.

In a preferred embodiment, apertures 22 are disposed such that the major axis 38 is at a right angle to the longitudinal axis 28 of lumen 18. The minor axis 40 is parallel to the longitudinal axis 28 of lumen 18. In the embodiment depicted in FIGS. 3 and 4, all apertures 22 are oriented such that major axis 38 is perpendicular to the longitudinal axis 28. However, in other embodiments, a selected number of apertures 22 may be in that orientation while other apertures are oriented in different directions. For example, apertures 22 may be oriented such that major axis 38 is perpendicular to longitudinal axis 28 at locations where the cannula 10 exhibits the greatest degree of bending during use in surgery, and therefore presents the greatest need for buckle-resistant apertures.

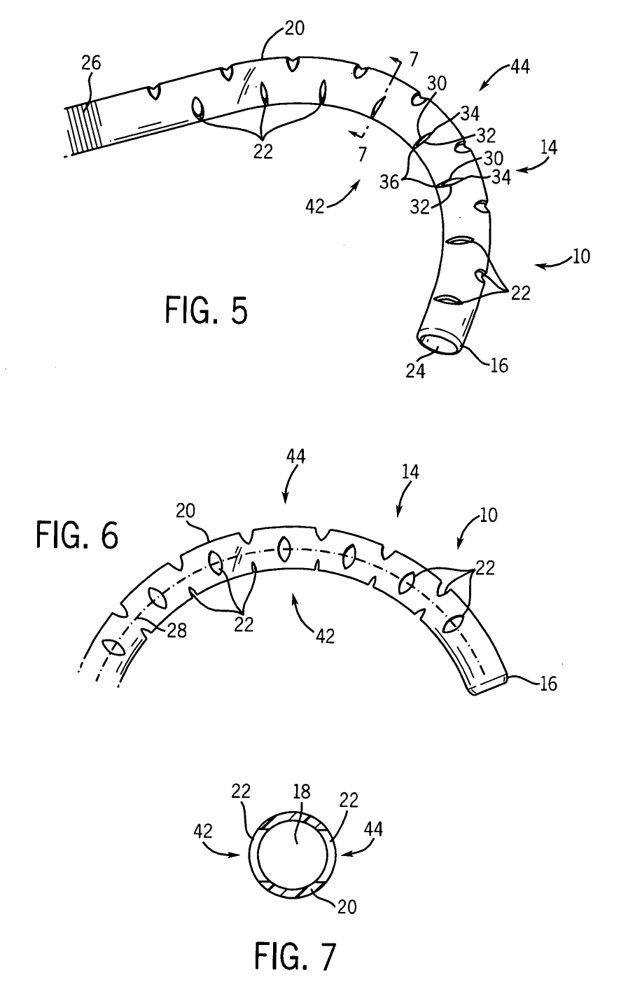

Referring to FIGS. 5 and 6, when cannula 10 is flexed, apertures 22 on the concave side 42 of flexed cannula 10 close to a certain degree in order to accommodate the extra wall material on the concave side 42. Apertures 22 on convex side 44 stretch open when cannula 10 is bent or flexed. Permitting apertures 22 to accommodate the extra wall material on concave side 42 by closing aids in preventing kinking of cannula 10 by taking up the stress in the tube wall 20 on the concave side of the body.

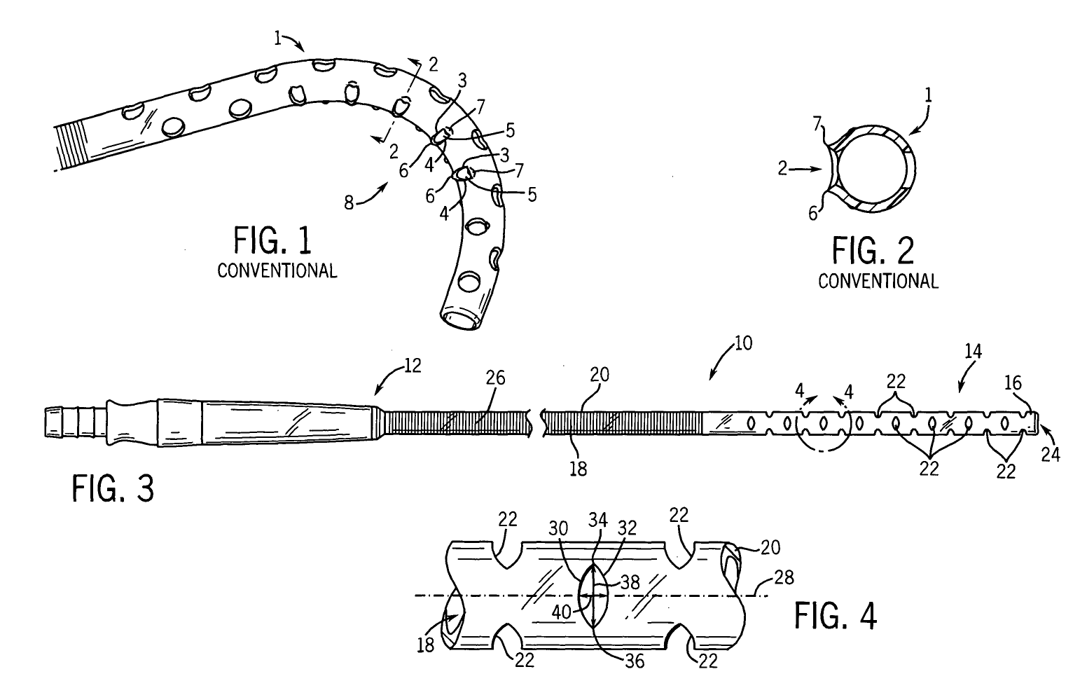

In the exemplary embodiment depicted in FIGS. 3-7, eye-shaped apertures 22 exhibit buckle resistant properties. The reduction in buckling is accomplished because first and second arcuate portions 30, 32 of each aperture 22 are able to close toward one another while the stress is taken up at corners 34, 36. Corners 34, 36 do not buckle, in contrast to the conventional design depicted in FIG. 1 where the aperture sides 6, 7 on the concave side 8 buckle when the cannula is flexed.

In an exemplary embodiment, apertures 22 are placed in wall 20 to preserve the structural integrity of cannula 10 when cannula 10 is flexed, while at the same time maintaining adequate flow characteristics. In the depicted embodiment, cannula 10 has four rows of several apertures extending along the longitudinal axis 28 of the lumen 18. The rows are evenly spaced such that each row is 90 degrees apart from adjacent rows and non-adjacent rows are 180 degrees apart from one another. The use of parallel but separated rows of lumens may permit the incorporation of barium stripes (not shown) in the cannula wall between the rows of apertures, the barium stripes being useful for X-ray imaging of the cannula while in the body. Adjacent rows are staggered such that apertures 22 are not placed immediately next to one another, each aperture 22 in adjacent rows being a different distance from tip 16. The staggered aperture placement increases the area of wall 20 between adjacent apertures 22, thus increasing structural integrity of the cannula body and therefore increasing resistance to kinking. In the exemplary embodiment, rows that are separated by 180 degrees have aligned apertures 22.

In other embodiments, the apertures may be placed on the cannula body in different patterns, such as in a spiral configuration or including more or fewer rows extending along the body. Further, the size of the individual apertures may differ from that depicted in the figures. Aperture placement may facilitate different functions of the cannula. For example, placing a set of apertures near the tip separated from a more proximal group of apertures by a continuous wall segment without apertures may create a dual stage cannula used to drain two portions of a patient's heart simultaneously. The continuous wall segment may extend a distance of approximately 1.75 inches along the cannula body between the sets of apertures. Further still, the size and placement of apertures differs depending on the use of the cannula, for example whether the cannula is a venous drainage cannula or an arterial perfusion cannula.

Cannula 10 may be made of various materials and manufactured by various methods. Exemplary materials include polyvinyl chloride (PVC), plastisol, and polyurethane. In a preferred embodiment, cannula 10 is made of polyurethane using an extrusion process. In the process, a first layer of the cannula wall is extruded. The reinforcement spring 26 may then be slipped over the first wall portion and followed by the extrusion of additional material over the top of the reinforcement spring 26 to enclose the reinforcement spring in the cannula wall. After the major steps are performed to create the wall 20 and reinforcement spring 26 structure, apertures 22 may be added. Another method of making a cannula is a dip-molding process using a mandrel dipped in a material such as plastisol or polyurethane.

After the cannula body is formed, apertures 22 are added into wall 20 to allow communication between the lumen 18 and the exterior of cannula 10 using a punch or drill process. Eye-shaped holes do not lend themselves to a drilling process so an eye-shaped punch may be used to add apertures 22 through cannula wall 20. In certain cases, the distal end of the cannula may be a separate piece (such as a portion with apertures, but without a reinforcing spring) that is attached to the proximal end of the cannula (having a reinforcing spring) at a later stage of the manufacturing process by a known process such as by RF welding.

In an exemplary embodiment, a method of punching apertures 22 minimizes the possibility of apertures 22 buckling when cannula 10 is flexed. In this embodiment, apertures 22 are punched into the concave side 42 of cannula 10 while cannula 10 is bent or flexed. An oval punch may be most suitable for this method. Apertures 22 may be punched individually such that each aperture 22 is punched while the cannula 10 has been flexed into the appropriate configuration (such as punching each aperture 22 at the apex of the concave side 42). The cannula is then straightened out and apertures 22 stretch somewhat as the concave side 42 wall material regains its original length. When cannula 10 is later flexed, such as during a surgical procedure, apertures 22 on the concave portion 42 of the curve assume the shape of the original punched holes rather than buckling as may occur in other designs.

When the above-described manufacturing method is utilized to create apertures, a punch used to create an oval or elliptical aperture may be suitable to minimize buckling without requiring an eye-shaped punch. Note that it may be preferable to punch one row of apertures at a time into the cannula wall along the concave portion of the flexed cannula to achieve best results.

The orientation of non-circular apertures such that the longer major axis of each aperture extends at a right angle to the lumen longitudinal axis may be advantageous as a feature used to minimize buckling with several shapes of apertures. While eye-shaped apertures are shown in FIGS. 3-6, other non-circular apertures also derive the benefit of the depicted orientation. For example, an oval aperture may exhibit reduced buckling tendencies when aligned such that the longer major axis is perpendicular to the longitudinal axis of the cannula. Further, an oval aperture may be sized to provide a similar flow rate to a similarly sized circular aperture while also deriving the benefit of reduced buckling due to the described orientation.

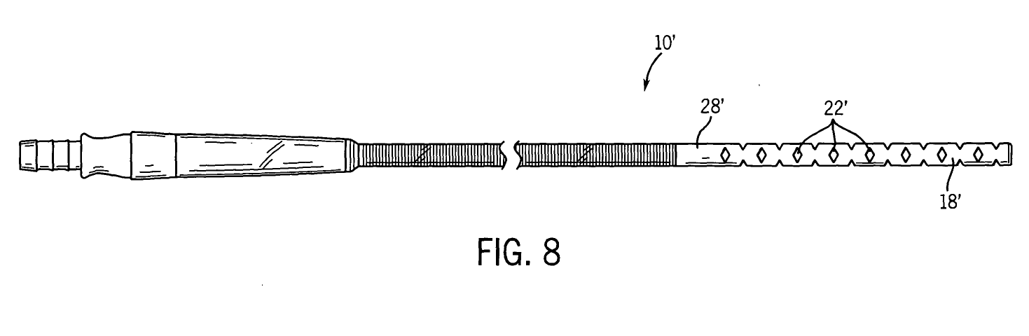

Referring to FIG. 8, in an exemplary embodiment, cannula 10′ includes diamond-shaped apertures 22′ that are oriented such that a longer major axis of each aperture 22′ is oriented at a right angle to the longitudinal axis 28′ of lumen 18′. Apertures 22′ are intended to exhibit buckle resistant properties and may be placed on the cannula wall in similar patterns to those described herein with respect to the other described cannula embodiments.

While the detailed drawings and specific examples given herein describe various exemplary embodiments, they serve the purpose of illustration only. It is to be understood that the invention is not limited in its application to the details of construction and arrangements of components set forth in the preceding description or illustrated in the drawings.

For example, while a venous cannula (which may or may not be vacuum-assisted) is shown incorporating the various aspects of the invention, the invention may also be applicable to arterial cannulae, cardioplegia cannulae, or other cannula or catheter designs that derive a benefit from reduced kinking and aperture buckling properties. Other examples may include femoral access cannulae and tubes used in neurological applications such as brain perfusion tubes. Such cannulae are available in many sizes, shapes, and lumen configurations (such as single and dual lumen) and are used in different types of surgical procedures. Furthermore, other substitutions, modifications, changes, and omissions may be made in the design, operating conditions, and arrangements of the exemplary embodiments without departing from the scope of the invention as expressed in the appended claims.

1. A cannula, comprising:
a body having a proximal end-and a distal end, the body having a wall defining a lumen extending from the proximal end to the distal end, the lumen having a longitudinal axis; and
a plurality of apertures in the wall interconnected with the lumen, wherein each of the apertures has a longer major axis and a shorter minor axis, and wherein the longer major axis is perpendicular to the longitudinal axis of the lumen.

2. The cannula of claim 1, wherein the cannula is a venous cannula.

3. The cannula of claim 1, wherein the apertures are eye-shaped.

4. The cannula of claim 1, wherein the apertures are oval.

5. The cannula of claim 1, wherein the apertures are a shape defined by first and second arcuate portions that intersect with one another at two corners.

6. The cannula of claim 1, wherein the apertures are arranged into a plurality of rows generally extending along the longitudinal axis of the lumen.

7. The cannula of claim 6, wherein the rows are evenly distributed on the body and the apertures of adjacent rows are offset such that the apertures in the adjacent rows are different distances from a distal tip of the body.

8. A cannula, comprising:
a body having a proximal end and a distal end, the body having a wall defining a lumen extending from the proximal end to the distal end, the lumen having a longitudinal axis; and
a plurality of apertures in the wall, wherein the apertures are eye-shaped.

9. The cannula of claim 8, wherein the cannula is a venous cannula.

10. The cannula of claim 8, wherein each of the apertures has a longer major axis and a shorter minor axis, and wherein the longer major axis is perpendicular to the longitudinal axis of the lumen.

11. The cannula of claim 10, wherein the apertures are a shape defined by first and second arcuate portions that intersect with one another at two corners.

12. The cannula of claim 8, wherein the apertures are arranged into four rows generally extending along the longitudinal axis of the lumen.

13. The cannula of claim 12, wherein the rows are evenly distributed on the body and the apertures of adjacent rows are offset such that the apertures in the adjacent rows are different distances from a distal tip of the body.

14. A method of making a cannula, comprising the steps of:
forming a cannula body having a wall defining a lumen;
bending the cannula body in a first direction such that the cannula body has a concave side and a convex side;
punching an oval aperture into the concave side of the body; and
straightening the cannula body.

15. The method of claim 14, wherein the wall is formed by extruding a plastic material.

16. The method of claim 15, wherein the plastic material is polyurethane.

17. The method of claim 14, wherein the body is formed by a dip molding process.

18. The method of claim 14, wherein the cannula is a venous cannula.

19. The method of claim 14, wherein the oval aperture has a longer major axis and a shorter minor axis, and wherein the longer major axis is perpendicular to a longitudinal axis of the lumen.

20. The method of claim 14, further comprising the step of punching a first row of oval apertures extending along the lumen into the concave side of the body before straightening the cannula body.

21. The method of claim 20, further comprising:
bending the cannula body in a second direction such that a different portion of the wall forms the concave side of the body; and
punching a second row of oval apertures extending along the lumen in the concave side of the body.

22. The method of claim 21, wherein the first and second rows are offset such that each aperture is a different distance from a distal tip of the body.
